(19) United States
(12) Patent Application Publication
(10) Pub. No.: US 2025/0041483 A1
(43) Pub. Date: Feb. 6, 2025

(54) GENETICALLY MODIFIED BIOPROSTHETIC HEART VALVE AND PRODUCTION METHOD THEREOF

(71) Applicant: KOC UNIVERSITESI, Istanbul (TR)

(72) Inventors: Kerem PEKKAN, Istanbul (TR); Hummaira Banu SIDDIQUI, Istanbul (TR); Merve CELIK, Istanbul (TR)

(73) Assignee: KOC UNIVERSITESI, Istanbul (TR)

(21) Appl. No.: 18/718,069

(22) PCT Filed: Dec. 9, 2021
(86) PCT No.: PCT/TR2021/051384

§ 371 Date: Jun. 10, 2024

(51) Int. Cl. A61L 27/36; C12N 15/113

(52) U.S. Cl. CPC A61L 27/3625; A61L 27/3687; A61L 27/3691; C12N 15/1136; A61L 2400/02; A61L 2430/20; C12N 2310/14

(57) ABSTRACT

A genetically modified bioprosthetic heart valve and a production method thereof are provided. The production method includes two critical steps for the invention to achieve its related objectives. These criteria are the molecular modification step and subsequent decellularization of animal-based heart valve in valve culture, where the molecular modification step involves an electroporation system and siRNA molecules.

### CROSS REFERENCE TO THE RELATED APPLICATIONS

This application is the national phase entry of International Application No. PCT/TR2021/051384, filed on Dec. 9, 2021, the entire contents of which are incorporated herein by reference.

### TECHNICAL FIELD

The present invention relates to a genetically modified bioprosthetic heart valve and more particularly a method for producing the same comprising two critical steps for the invention to achieve its related objectives. These criteria are the molecular modification step and subsequent decellularization of animal-based heart valve in valve culture.

### BACKGROUND

In general, prosthetic heart valves can categorized into three types: mechanical heart valves, bioprosthetic heart valves, and homograft valves. Bioprosthetic heart valves are composed of three xenograft tissue leaflets of either porcine valvular leaflets or bovine pericardial tissue [1].

Bioprosthetic heart valves studies focus on four criteria: hemodynamics, mechanical and biological durability, minimizing immune response or rejection, and delivery system [1].

In current aortic valve implantations, only decellularization is carried out which does not have the potential of eliminating some structural issues which cause complications in the patients.

According to the literature, decellularization processes have been mentioned and used for valves. It is reported that current tissue engineering strategies in fabricating heart valves and their progress towards the clinic, including molded scaffolds using naturally derived or synthetic polymers, decellularization, electrospinning, 3D bioprinting, hybrid techniques, and in vivo engineering [2].

In addition, heart valve disease is characterized by the loss of the highly organized structure of the valves, which results in the alteration of the mechanical properties. The structural integrity is therefore critical for the function of the valve. The molecular modification will attempt to produce a functional valve structure and to eliminate these post-operative complications.

Molecular modification step has also been attempted so as to eliminate the functional problems of the valve [3, 4]. However, according to our invention the use of molecular modification preceding decellularization is novel and has never been attempted.

The use of only decellularization creates problems post-operation such as calcification and incompatibility in structure [5]. The use of only molecular modification causes issues such as introduction of non-human pathogens and the severe immune responses [6]. Combining the two processes creates a solution which encompasses the advantages of both and simultaneously eliminates the disadvantages of both the procedures.

### SUMMARY

The present invention relates to a bioprosthetic heart valves and a method of producing the same.

The present invention provides for bio-engineered, or tissue engineered bioprosthetic heart valves that have a decreased inflammatory potential.

The present invention aims at targeting certain cells (such as valvular interstitial and valvular endothelial cells) which are responsible for causing desired structural changes in the aortic valve, to alter these characteristics as required.

The present invention is also aimed to compare the newly produced aortic valve with commercially available valves and unmodified valves in terms of biological, physical, and mechanical properties.

The present invention overcomes the deficiencies of previous problems mentioned above and provides distinct advantages over the prior art. Generally, the present invention provides a method for producing bioprosthetic aortic heart valves for tissue engineering applications by altering the physical attributes of the aortic heart valve and customizing it according to patients' demands.

Another aspect of the present invention relates to a method for producing bioprosthetic aortic heart valves to eliminate the immune rejection of implanted valves and to improve current clinically used valves. This would benefit patients (especially pediatric patients) who require a heart valve implantation due to severe defects in their own valves. This method ensures a minimally invasive method of operation, an adaptable bioprosthetic that doesn't need constant replacement and a reduction in complications which are observed in patients that undergo heart valve implantation.

This object and other objects of this invention become apparent from the detailed discussion of the invention that follows.

### BRIEF DESCRIPTION OF THE DRAWINGS

“Genetically Modified Bioprosthetic Heart Valve and a Method of Producing the Same” developed to fulfill the objects of the present invention is illustrated in the accompanying figures wherein

FIG. 1 shows the silenced mechanosensitive gene TGFβ-3 which is presented according to the siRNA delivery system with our in-house in vivo electroporation.

FIG. 2 shows the scheme of the method. The successful knockout of genes responsible for calcification has been done in our lab seen in FIG. 2.

### DETAILED DESCRIPTION OF THE EMBODIMENTS

The present invention relates to a genetically modified bioprosthetic heart valve and more particularly a method for producing the same comprising two critical steps for the invention to achieve its related objectives. The invention further relates to the method for producing bioprosthetic heart valves to reduce in vivo calcification.

According to the present invention, these critical steps of the method are the molecular modification step and subsequent decellularization of animal-based heart valve in valve culture. More preferably, according to the invention molecular modification is applied preceding decellularization.

The term “produce” or “producing” used herein refers to preparing and making. More preferably, the method of producing means preparing the tissue, organ, or cells to the implant application with overcoming the issues mentioned above (e.g., structural, functional issues, immune response of the body) which may cause complications in the patients. According to the type of implant, if the patient is human, the initial implant tissue, organ or cell is from non-human origin(s). Preferably, in this invention the patient or recipient is human.

The targeted cells, organs or tissue are immunologically compatible with the intended implant recipient. The valves produced by this method is free from many of the disadvantages of other prior art bioprosthetic heart valves.

In a preferred embodiment of the invention, the animal-based heart valve is porcine based, bovine based (bovine pericardium) or combination thereof. In the literature, it is also mentioned as xenografts which should be cleaned, sterilized, and prepared for implantation. Bovine and porcine based heart valve tissues are structurally and bio-mechanically appropriate for use in humans. However, introduction of such foreign tissues can trigger the immune response or rejection, and post-operational complications can be occurred.

Moreover, these xenografts are nonviable, so they tend to calcify with time because of the lack of any repair mechanism. Calcification of the heart valve tissue can increase the risk of structural damage and even failure. Calcification can cause alterations such as increasing the thickness of the bioprosthetic heart valve and altering its mechanical properties, which prevents their proper functioning and can cause their rupture. The present invention overcomes all these problems.

In accordance with the purpose(s) of the invention, as embodied and broadly described herein, the present invention relates to a novel method for preparing a genetically modified bioprosthetic heart valve, the method comprises:
providing an aortic heart valve from the animal most suitable for implantation
treatment of the aortic heart valve by a molecular modification process
decellularization of the aortic heart valve,
   wherein molecular modification is applied preceding decellularization.

According to the present invention, the first step simply requires the procurement of the aortic heart valve from the animal most suitable for implantation. Research shows that the bovine valves are the most suitable for the same [1, 14]. Preparation of the aortic valve culture will also be done in this step. For the preparation, the valves are to be stored in glutaraldehyde solution with a pH of 7.2 to 7.4 at 3° C. These valves can be stored for up to 7 months. At −40° C., the valves can be stored for longer in formaldehyde solution. Procurement of the aortic heart valve can be performed by extraction from sheep and transportation of extracted aortic heart valve in appropriate and aseptic conditions.

In the second step, the treatment of the valve can be done with various molecules for various methods. The molecules have been studied for their properties and have been selected according to desired changes. According to the invention, the molecular modification step comprises:
a) Cultivation of aortic heart valve in organ culture system
b) Gene level modification using our in-house electroporation system and re-culture.

Heart valve calcification is associated with inflammation which accelerates the progression of calcification. According to the present invention, the related target molecules mentioned in the second step are associated with heart valve calcification problem. For example, receptor activator of the nuclear factor KB ligand (RANKL), Beta-catenin (β-catenin), and Transforming Growth Factor-β (TGF-β) are proposed to play central roles in the calcification of heart valves. Molecules such as bone morphogenetic protein 2 (BMP2), Smad1, and MSX2 are targeted by the given genes. ELN, FBN-1, COL I and COL III are downregulated by TGF-β Genes such as ASCC3, ITK, CD28, and LINS are also specific to aortic valve calcification [16]. Nox2, MMP-9, VCAM1, MMP9, ITGB2, RAC2, vWF and ALDH2, played a role in formation of calcified aortic valve [17].

The molecular modification step provides an electrophoretic system and method of use thereof for the preparation of a genetically modified bioprosthetic heart valve. The gene level modification step comprises an agent which is used for reducing, suppressing, or silencing gene or messenger RNA (mRNA) expression, or protein expression. Such agents include nucleic acids, small interfering RNA (siRNA), and antisense RNA and the like. More preferably, siRNA is used in the present invention.

The third step, following the molecular modification step is called decellularization process. According to the invention, the decellularization process can be done in one of two ways: immersion or perfusion. Each of these done using predetermined chemical compounds such as dodecyl sulfate, trypsin, ethylenediamine, tetra acetic acid, triton X-100, sodium deoxycholate etc. [2]. The sterilization process would be the preparation for the implantation. In this step, confirmation of decellularization by DNA content measurement and histological analysis can be performed.

In one embodiment of the present invention, after the decellularization process, the tests for the success of the modification are performed and a comparative analysis of native and modified aortic heart valve is conducted. The bioprosthetic heart valve is safely stored till implantation.

Preferably, in the clinical use the transport of the valve from the lab to the hospital can be facilitated.

The method according to the present invention is a kind of treatment process for bioprosthetic tissues used in implants such as bioprosthetic heart valves to reduce in vivo calcification of the valve. The method described in this invention is for preparing improved bioprosthetic heart valves for human (or mammalian) implant, by the treatment process of nonhuman valve tissue.

References [7] and [8] prove that calcification is reduced when KO (knockout) is performed. References [9], [10], [11], and are for other KO experiments that have been successfully conducted for xenotransfusion.

These examples are intended to representative of specific embodiments of the invention and are not intended as limiting the scope of the invention.

### SPECIFIC EMBODIMENTS

Experimental studies lead to determine the advantages of the present invention.

### EXAMPLES

### Example 1 Overview

The experiments will be started first by selecting a case. For this project, the calcification of the valve will be targeted.

Heart valve calcification is associated with inflammation which accelerates the progression of calcification. For example, receptor activator of the nuclear factor KB ligand (RANKL), Beta-catenin (β-catenin), and Transforming Growth Factor-β (TGF-β) are proposed to play central roles in the calcification of heart valves.

Herein, Wnt RANK/RANKL, Wnt β-catenin, and TGF-β3 were and Beta-catenin used for the molecular modification of the valve. This step would take a few weeks (Approximately 6 weeks). In the following month, decellularization will be done using ethylene diamine or trypsin to ensure minimum damage to the scaffold. The optimization of this step could take about 6-8 weeks. Once these steps are completed, the valves will be tested by simulating implantation and observing the difference between unmodified valves and the modified valves.

### Example 2 Molecular Modification Step

According to the invention, the molecular modification step comprises:
a) Cultivation of aortic heart valve in organ culture system
b) Gene level modification using our in-house electroporation system and re-culture.

### Materials:

siRNA-Cy5 solution
Microscope.
Microinjection system.
Electroporation system (custom designed).
Chick Embryo at HH18
Microneedle (custom designed).
Pressure manipulator

To culture heart valves, the protocol from the following reference can be applied [18]. The electroporation system unique to this patent is described below.

### Method:

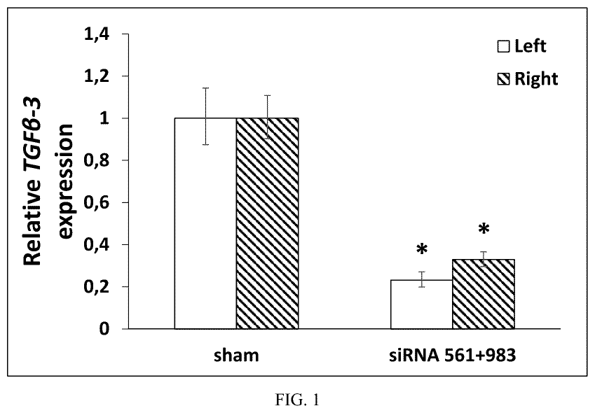

Using our micro-electroporation system, silencing or overexpression of certain molecules will be targeted in the aortic valve depending on the signal pathway. With this system, we performed the specific siRNA molecules delivery through micro needle and conducted electroporation.
1. A fertilized chick embryo was obtained from the farm and incubated at 37° C. and 65% humidity until it reaches HH18
2. A small window is opened in the egg to access the embryo
3. A microneedle was attached to a pneumatic pressure system and loaded with 20 microliters of specific siRNA
4. The siRNA-Cy5 solution (negative control) was injected into the aortic arch region of chick embryo by adjusting the pressure in the needle using the pneumatic pressure system.
5. The electrodes of the electroporation system are placed around the target region.
6. The Optimized parameters for the electroporation system are 10 square wave pulses (amplitude: 14V p-p, period: 100 ms, 50% duty cycle).
7. Using the LabView software an electric pulse with desirable potential difference is applied for approximately one second.
8. The microneedle and the electrodes are removed and the egg shell of the chick embryo is covered using paraffin film and is placed back in the incubator for development.
9. Among several genes successfully altered in live embryo, the results of silenced mechanosensitive gene TGFβ-3 are presented (FIG. 1).

The valve is to be obtained from farms which breed animals for slaughter and hence all ethical dilemmas can be avoided. The culture medium for the valve during transport and modification is to be determined and due to abundance of information, this step has various options that can be chosen from.

### Example 2 Decellularization Step

First Decellularization protocol: (Schmidt et al., 2007)

### Preparation of Antimicrobial Solution.

1.2 mg amikacin
3 mg flucytosine.
1.2 mg vancomycin.
0.3 mg ciprofloxacin.
1.2 mg metronidazole in 1 ml aqua ad inject

### Preparation of Decellularization Solution.

0.05% trypsin for enzyme lysis.
0.02% EDTA to deactivate metal dependent enzymes

### Protocol

1. Reduce time between slaughter and to reduce autolysis.
2. Wash the valve for 30 mins at room temperature in PBS solution and povidone-iodide solution
3. Treat for 12 hours in antimicrobial solution
4. Incubate at 37° C. and 5% CO2 for 24 hours under 3D shaking
5. Rinse again with PBS for 24 hours to wash away the cell detritus
6. Store in hanks balance salt solution at 4° C. till the time of further experimentation

### Second Decellularization Protocol:

### Prepare a Decellularization Solution:

0.05% sodium dodecyle sulfate (SDS) helps in lysing cells by disrupting non-covalent bonds and hence degrading the proteins in the cells.
0.05% sodium deoxycholate (SDCh) helps in emulsification of fats. Isolates membrane associated proteins

### Detergent Treatment:

1. The process must take place under germ reduced conditions. Separate the valve from the rest of the tissues surrounding it and store at 4° C. for 24 hours in distilled water. (Need for this step)
2. Treat the gathered valve with the decellularization solution for 24 hours by immersion.
3. Rinse the valve with distilled water for 24 hours
4. Treat the rinsed valve with phosphate buffered saline solution (PBS) and antibodies such as penicillin and streptomycin and partricin (antimyocotics) for thorough cleansing.

### Results:

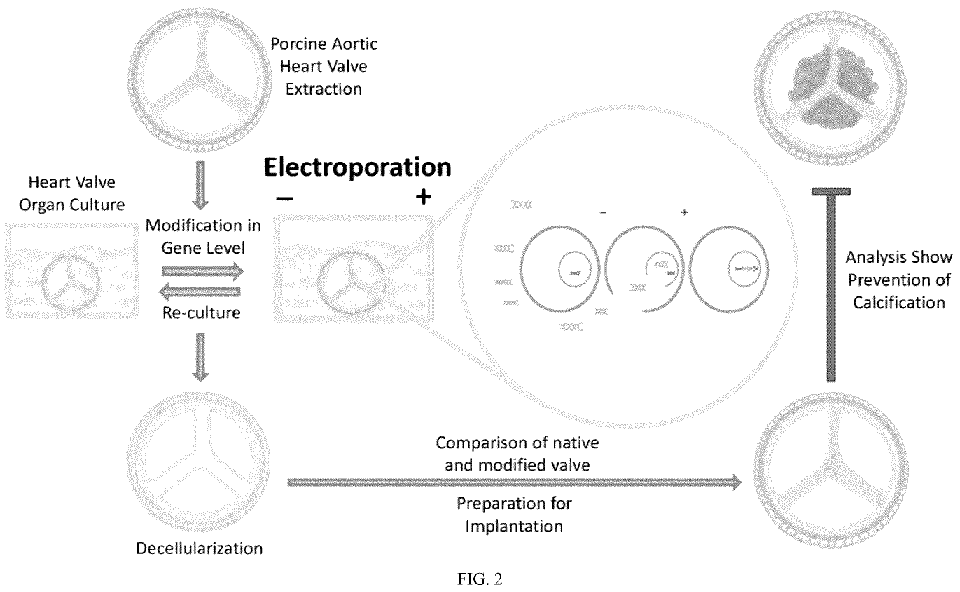

The successful knockout of genes responsible for calcification has been done in our lab seen in FIG. 2. This knockout was performed by using TGFb3 siRNA and electroporation as illustrated in FIG. 1.

Literature shows evidence of successful prevention of calcification due to knockout [7, 8]. Decellularization aims to reduce immune rejection of the implanted valve. While the current status of the experiment has reached the knockout stage, decellularization steps are underway to complete the experiment successfully. Bovine pericardium tissues are being used to perform knockout and decellularization to create the most biocompatible valve.

### REFERENCES

1. Pibarot, P. and J. G. Dumesnil, Prosthetic heart valves: selection of the optimal prosthesis and long-term management. Circulation, 2009. 119 (7): p. 1034-48.
2. Cheung, D. Y., B. Duan, and J. T. Butcher, Current progress in tissue engineering of heart valves: multiscale problems, multiscale solutions. Expert Opin Biol Ther, 2015. 15 (8): p. 1155-72.
3. Dasi, L. P., et al., Fluid mechanics of artificial heart valves. Clin Exp Pharmacol Physiol, 2009. 36 (2): p. 225-37.
4. Sewell-Loftin, M. K., et al., EMT-inducing biomaterials for heart valve engineering: taking cues from developmental biology. J Cardiovasc Transl Res, 2011. 4 (5): p. 658-71.
5. Schmidt, D., U. A. Stock, and S. P. Hoerstrup, Tissue engineering of heart valves using decellularized xenogeneic or polymeric starter matrices. Philos Trans R Soc Lond B Biol Sci, 2007. 362 (1484): p. 1505-12.
6. Yacoub, M. and R. Nerem, Introduction. Bioengineering the heart. Philos Trans R Soc Lond B Biol Sci, 2007. 362 (1484): p. 1253-5.
7. Smood, B., et al., In Search of the Ideal Valve: Optimizing Genetic Modifications to Prevent Bioprosthetic Degeneration. Ann Thorac Surg, 2019. 108 (2): p. 624-635.
8. McGregor, C. G., et al., Cardiac xenotransplantation technology provides materials for improved bioprosthetic heart valves. J Thorac Cardiovasc Surg, 2011. 141 (1): p. 269-75.
9. Lee, W., et al., Initial in vitro studies on tissues and cells from GTKO/CD46 NeuGckO pigs. Xenotransplantation, 2016. 23 (2): p. 137-50.
10. Lutz, A. J., et al., Double knockout pigs deficient in N-glycolylneuraminic acid and galactose alpha-1,3-galactose reduce the humoral barrier to xenotransplantation. Xenotransplantation, 2013. 20 (1): p. 27-35.
11. Wang, Z. Y., et al., Erythrocytes from GGTA1/CMAH knockout pigs: implications for xenotransfusion and testing in non-human primates. Xenotransplantation, 2014. 21 (4): p. 376-84.
12. Burlak, C., et al., Reduced binding of human antibodies to cells from GGTA1/CMAH KO pigs. Am J Transplant, 2014. 14 (8): p. 1895-900.
13. Iwase, H., et al., Regulation of human platelet aggregation by genetically modified pig endothelial cells and thrombin inhibition. Xenotransplantation, 2014. 21 (1): p. 72-83.
14. Butcher, J. T., G. J. Mahler, and L. A. Hockaday, Aortic valve disease and treatment: the need for naturally engineered solutions. Adv Drug Deliv Rev, 2011. 63 (4-5): p. 242-68.
15. Hu, X. J., et al., Role of TGF-beta1 Signaling in Heart Valve Calcification Induced by Abnormal Mechanical Stimulation in a Tissue Engineering Model. Curr Med Sci, 2018. 38 (5): p. 765-775.
16. MacGrogan, D., et al., Identification of a peripheral blood gene signature predicting aortic valve calcification. Physiol Genomics, 2020. 52 (12): p. 563-574.
17. Zhang, Y. and L. Ma, Identification of key genes and pathways in calcific aortic valve disease by bioinformatics analysis. J Thorac Dis, 2019. 11 (12): p. 5417-5426.
18. Kruithof, B. P., et al., Culturing Mouse Cardiac Valves in the Miniature Tissue Culture System. J Vis Exp, 2015 (105): p. e52750.

What is claimed is:

1. A method for preparing a genetically modified bioprosthetic heart valve, comprising:
providing an aortic heart valve from an animal most suitable for an implantation,
treating the aortic heart valve by a molecular modification process,
performing a decellularization of the aortic heart valve,
wherein the molecular modification process is applied preceding the decellularization.

2. The method according to claim 1, wherein the molecular modification process comprises an electroporation system.

3. The method according to claim 2, wherein the molecular modification process comprises at least one small interfering RNA (siRNA) molecule.

4. The method according to claim 3, wherein a target molecule of the at least one siRNA molecule is selected from molecules associated with a heart valve calcification problem.

5. The method according to claim 4, wherein the target molecule is at least one selected from the group consisting of receptor activator of the nuclear factor κB ligand (RANKL), Beta-catenin (β-catenin), Transforming Growth Factor-β (TGF-β), bone morphogenetic protein 2 (BMP2), Smad1, MSX2, ELN, FBN-1, COL I, COL II, ASCC3, ITK, CD28, LINS, Nox2, MMP-9, VCAM1, MMP9, ITGB2, RAC2, vWF, and ALDH2.

6. The method according to claim 5, wherein the target molecule is at least one selected from the group consisting of the RANKL, the β-catenin, and the TGF-β.
